(19) United States
(12) Patent Application Publication
(10) Pub. No.: US 2011/0144662 A1
(43) Pub. Date: Jun. 16, 2011

(54) CLIP DEVICES AND SYSTEMS AND METHODS FOR DEPLOYMENT

(76) Inventors: Tyler E. McLawhorn, Winston-Salem, NC (US); Richard W. Ducharme, Winston-Salem, NC (US); Vihar C. Surti, Winston-Salem, NC (US); Michelle D. Martinez, Winston-Salem, NC (US)

(21) Appl. No.: 12/964,920

(22) Filed: Dec. 10, 2010

Related U.S. Application Data
(60) Provisional application No. 61/286,673, filed on Dec. 15, 2009.

(51) Int. Cl. A61B 17/10; A61B 17/08

(52) U.S. Cl. 606/142; 606/151

(57) ABSTRACT

The present embodiments provide clip devices, along with systems and methods for delivering and deploying the clip devices. An exemplary clip device comprises inner and outer segments, and further has a delivery configuration, a tissue receiving configuration and a deployed configuration. A distal region of the outer segment is movable with respect to a distal region of the inner segment, thereby creating a spacing between the inner and outer segments for surrounding tissue in the tissue receiving configuration. The inner and outer segments may be biased to return to the deployed configuration, and may comprise generally identical shapes in the deployed configuration wherein the outer segment is proportionally larger than the inner segment. In one embodiment, the inner and outer segments each comprise “V” shapes in the unbiased state. Various delivery systems and methods are provided for delivering and deploying the clip devices disclosed.

### PRIORITY CLAIM

This invention claims the benefit of priority of U.S. Provisional Application Ser. No. 61/286,673, entitled “Clip Devices and Systems and Methods for Deployment,” filed Dec. 15, 2009, the disclosure of which is hereby incorporated by reference in its entirety.

### BACKGROUND

The present invention relates to a clip device, and more specifically, to a clip device that can be used for holding tissue or the like, and systems and methods for the deployment of the clip device.

During medical procedures it may be necessary or desirable to apply various forces upon tissue. As one example, during a laparoscopic cholecystectomy, a physician will occlude the common bile duct and artery towards the base of the gallbladder to prevent any leakage of bile into the peritoneum. An occluding device may comprise a mechanical device, such as a clip, or an electrical device. Various clips are known, including vascular clips, hemostatic clips, endoscopic clips, and surgical clips.

If a laparoscopic approach is employed, the mechanical or electrical devices may be delivered through a relatively short, rigid delivery trocar. The successful delivery of a clip or other device may rely on a proper selection of the trocar insertion location, among other factors.

When a conventional clip is delivered through a trocar, the width of the clip generally is limited by the inner diameter of the trocar. Therefore, in previous systems, a relatively wide clip cannot be delivered unless the width of the trocar is increased, which oftentimes is an undesirable result. If relatively wide clips cannot be provided due to the trocar size, then multiple clips may be needed to achieve proper sealing of the targeted duct or vessel, which may increase the complexity and time of the procedure.

Similarly, in situations where a laparoscopic cholecystectomy or other procedure is performed endoscopically instead of laparoscopically, for example, during a translumenal procedure, the size of the clip may be limited by the size of the lumen of an endoscope. In such a translumenal procedure, the endoscope may be inserted through a visceral wall, such as the stomach wall. Due to limitations associated with the diameter of the lumen of the endoscope, multiple relatively small clips may be needed to achieve proper sealing of the targeted duct or vessel.

### SUMMARY

The present embodiments provide clip devices, along with systems and methods for delivering and deploying the clip devices. An exemplary clip device comprises inner and outer segments, and further has a delivery configuration, a tissue receiving configuration and a deployed configuration. A distal region of the outer segment is movable with respect to a distal region of the inner segment, thereby creating a spacing between the inner and outer segments for surrounding tissue in the tissue receiving configuration. The inner and outer segments may be biased to return towards the deployed configuration in which they impose a compressive force upon tissue. In an unbiased configuration, the inner segment may be generally nestled laterally within the outer segment.

In one embodiment, the inner and outer segments of the clip device may comprise generally identical shapes in the deployed configuration, wherein the outer segment is proportionally larger than the inner segment. In one example, the inner segment and the outer segment each comprise “V” shapes in the deployed state.

An exemplary delivery device for use with the clip device comprises a tubular member having first and second pathways separated by at least one guide member. The delivery device further comprises a first ramp member positioned at the distal end of the first pathway, wherein the first ramp member is angled with respect to a longitudinal axis of the delivery device. The first ramp member is configured to guide the inner segment towards a desired angle in the tissue receiving configuration. Optionally, a second ramp member may be positioned at the distal end of the second pathway, wherein the second ramp member is angled with respect to the longitudinal axis to guide the outer segment towards a desired angle in the tissue receiving configuration.

In one method of operation, the clip device is loaded into the delivery device with the inner segment positioned at least partially within the first pathway and the outer segment positioned at least partially within the second pathway. The clip device is advanced distally with respect to the delivery device to cause the clip device to achieve the tissue receiving configuration in which a distal region of the outer segment is spaced apart from a distal region of the inner segment. In a next step, tissue is positioned between the inner and outer segments. Then, the clip device is deployed from the delivery device to cause the inner and outer segments to move towards each other and impose a compressive force upon tissue in the deployed configuration.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is a top view of a clip device according to a first embodiment in an unbiased configuration.

FIG. 2 is a top view of the clip device of FIG. 1 in a delivery configuration.

FIG. 3 is a top view of a delivery device according to a first embodiment.

FIG. 4 is side-sectional view of the delivery device of FIG. 3.

FIG. 5 is a side-sectional view of an alternative delivery device.

FIGS. 6-9 depict one exemplary method of use of the clip device of FIGS. 1-2 using the delivery system of FIGS. 3-4, with FIGS. 6-7 being shown from side-sectional views and FIGS. 8-9 being shown from top views for illustrative purposes.

FIG. 10 is a side-sectional view of a further alternative delivery device.

FIG. 11 is a side view of a clip device used with the delivery device of FIG. 9.

FIG. 12 is a top view of an alternative clip device in a deployed configuration.

FIG. 13 is a top view of a clip device according to an alternative embodiment in an unbiased configuration.

FIG. 14 is a perspective view of the clip device of FIG. 13 in a tissue receiving configuration.

### DETAILED DESCRIPTION OF THE PREFERRED EMBODIMENTS

In the present application, the term “proximal” refers to a direction that is generally towards a physician during a medical procedure, while the term “distal” refers to a direction that is generally towards a target site within a patent's anatomy during a medical procedure.

Referring to FIG. 1, a first embodiment of a clip device 20 is shown. The clip device 20 has proximal and distal regions 22 and 24, respectively, and generally comprises an inner segment 30 and an outer segment 50. The inner segment 30 is separated from the outer segment 50 by a slit 75, as shown in FIG. 1. In one embodiment, in an unbiased configuration as shown in FIG. 1, the inner segment 30 is generally nestled laterally within the outer segment 50.

The clip device 20 generally is characterized by three dimensions, specifically, the x-y-z dimensions shown in FIG. 1. In the x-dimension, the clip moves longitudinally (e.g., left-to-right with respect to a piece of paper). In the y-dimension, the clip moves laterally (e.g., towards upper and lower surfaces of a piece paper). In the z-dimension, the clip moves vertically (e.g., in and out of a page with respect to a piece paper). In the unbiased configuration, the inner segment 30 and the outer segment 50 are generally in the same plane in the x-dimension.

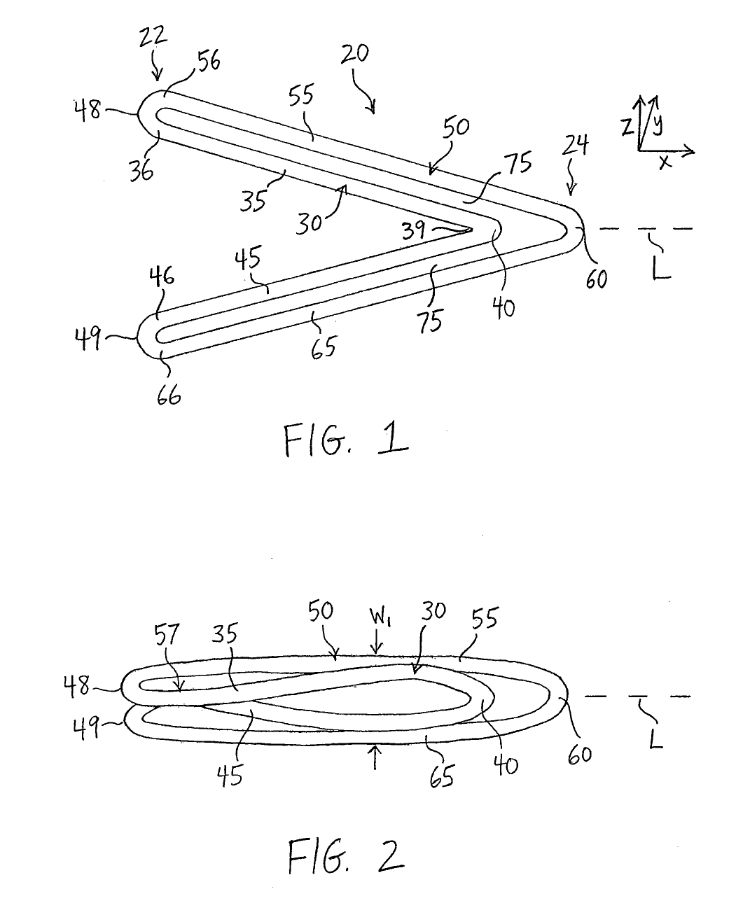

In the embodiment of FIGS. 1-2, the inner segment 30 and the outer segment 50 each comprise “V” shapes in the deployed state, and the slit 75 separating the inner and outer segments 30 and 50 further comprises a “V” shape. However, in alternative embodiments, the inner and outer segments 30 and 50 may comprise other shapes, including but not limited to a “U” shape (as shown in FIGS. 13-14 below), “W” shape, and the like, without departing from the spirit of the present invention.

The inner segment 30 has first and second regions 35 and 45 separated by a distal apex 40, thereby forming the “V’ shape in the deployed state shown in FIG. 1. The first region 35 of the inner segment 30 has a proximal portion 36 and a distal end that transitions into the apex 40, while the second region 45 had a proximal portion 46 and a distal end that also transitions into the apex 40.

Similarly, the outer segment 50 has first and second regions 55 and 65 separated by a distal apex 60, thereby forming the “V” shape in the deployed state shown in FIG. 1. The first region 55 of the outer segment 50 has a proximal end 56 and a distal end that transitions into the apex 60, while the second segment 65 had a proximal end 66 and a distal end that also transitions into the apex 60.

In the deployed “V” shape of FIG. 1, the proximal region 22 of the clip device 20 has first and second proximal apices 48 and 49. The first proximal apex 48 is a curved transition between the proximal portion 36 of the inner segment 30 and the proximal end 56 of the outer segment 50, while the second proximal apex 49 is a curved transition between the proximal portion 46 of the inner segment 30 and the proximal end 66 of the outer segment 50, as shown in FIG. 1. In this embodiment, the apices 48 and 49 generally serve as hinges between the inner and outer segments 30 and 50, although other hinge designs may be used such as external hinges coupled between the inner and outer segments 30 and 50 to achieve the same functionality described herein.

The clip device 20 may be made from any suitable resilient material, including but not limited to stainless steel, plastic, a shape-memory material such as nitinol, and the like. The clip device 20 may be moved between the deployed configuration of FIG. 1 and a delivery configuration, as shown in FIG. 2. In the absence of a significant external force acting up the clip device 20, the inner and outer segments 30 and 50 are biased to return to the unbiased configuration of FIG. 1.

The clip device 20 may be moved between the unbiased configuration of FIG. 1 and the delivery configuration of FIG. 2 by applying a compressive to bring the first and second proximal apices 48 and 49 laterally closer to one another, i.e., permitting movement in the y-dimension. In one example, the first and second regions 55 and 65 of the outer segment 50 may be compressed, manually or using an automated device, thereby urging the clip device 20 to assume the delivery configuration shown in FIG. 2. In the delivery configuration, the first region 35 of the inner segment 30 and the first region 55 of the outer segment 50 are moved towards the second region 45 of the inner segment 30 and the second segment 65 of the outer segment 50 to achieve a reduced profile. In particular, the first and second proximal apices 48 and 49 may be positioned adjacent to one another, or partially or fully overlapping one another. Preferably, a narrowed region 57 of the inner segment 30 may be formed upon compression of the inner segment 30. The narrowed region 57 may comprise an area of partial or complete overlap of the first and second regions 34 and 45, which may facilitate positioning of the clip device 20 around guide members within a delivery device 80, as explained further in FIG. 6 below.

In the delivery configuration of FIG. 2, an overall width of the clip device 30 is reduced relative to the unbiased configuration of FIG. 1. For example, by bringing the first and second proximal apices 48 and 49 adjacent to one another, or overlapping one another, the width of the clip device may be reduced by a factor of about 3-5 times. Advantageously, as will be explained in further detail below, the clip device 20 therefore may be delivered via a relatively small insertion device, and then expanded to increase its surface area engagement with tissue.

Notably, when compressed in the delivery configuration, at least a portion of the inner segment 30 and the outer segment 50 may be moved into a different plane relative to one another. In particular, the distal apex 40 of the inner segment 30 may move apart from the distal apex 60 of the outer segment 50 in the z-dimension, as depicted from a top view in FIG. 2 and from a side-sectional view in FIG. 6 below. As will be explained further below, by forming a spacing between distal regions of the inner and outer segments 30 and 50, tissue may be received between the inner and outer segments 30 and 50 as depicted with respect to FIGS. 7-8 below.

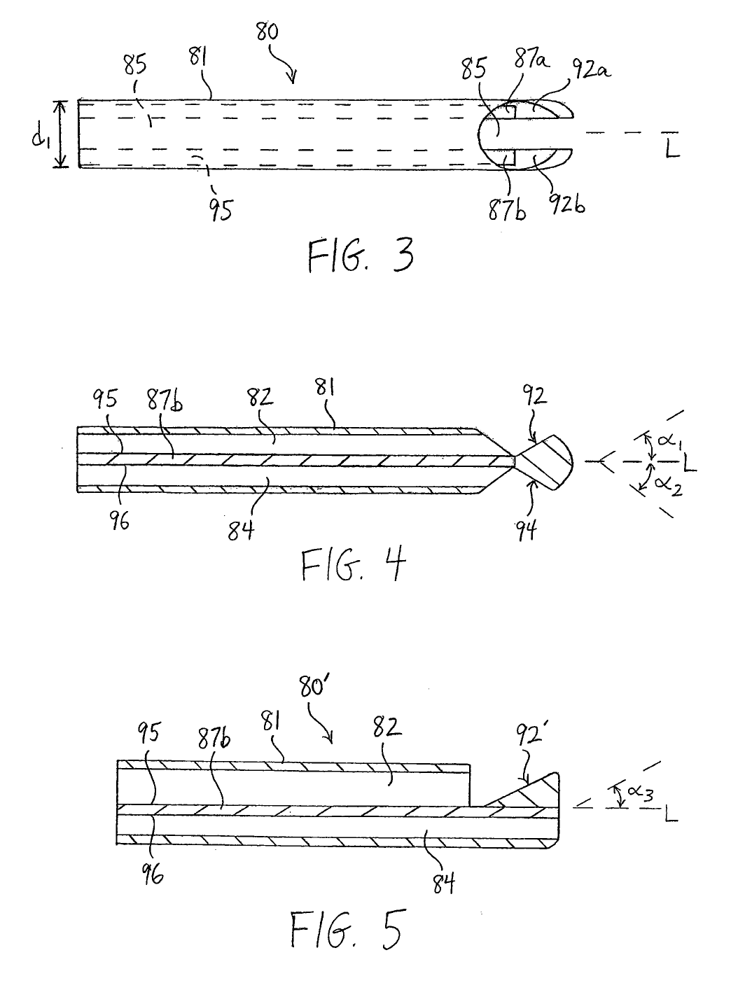

Referring now to FIGS. 3-4, a first embodiment of a delivery device 80 suitable for delivering the clip device 20 is shown from top and side-sectional views, respectively. The delivery device 80 comprises a tubular member 81 having proximal and distal regions. In one embodiment, the tubular member 81 is formed from a thin-walled cannula, such as a metal cannula. However, any suitable material may be used, including plastics (e.g., a catheter), and the tubular member 81 may be formed by extrusion or other suitable techniques.

The tubular member 81 comprises first and second pathways 82 and 82, as best seen in FIG. 4. In this embodiment, first and second guide members 87a and 87b protrude inward from an interior wall of the tubular member 81, as shown in FIG. 3. The first and second guide members 87a and 87b may be disposed about 180 degrees apart around the inner circumferences of the tubular member 81, and are spaced apart by a lumen 85, thereby forming a set of opposing guides, as best seen in FIG. 3. The provision of the first and second guide members 87a and 87b generally separates the first and second pathways 82 and 84 within the tubular member 81. Both guide members 87a and 87b have a first surface 95 that faces towards the first pathway 82 and a second surface 96 that faces towards the second pathway 84. For example, the first region 35 of the inner segment 30 rides above guide member 87a, the first region 55 of the outer segment 60 rides below guide member 87a, the second region 45 of the inner segment 30 rides above guide member 87b, and the second region 65 of the outer segment 60 rides below guide member 87b, as explained further below.

In the embodiment of FIGS. 3-4, the delivery device 80 further comprises first and second ramp members 92 and 94. The first ramp member 92 comprises first and second ramp guides 92a and 92b, respectively, which are spaced apart by the lumen 85, as depicted in FIG. 3. The first and second guide members 87a and 87b extend distally such that their upper surfaces 95 transition into the first and the second ramp guides 92a and 92b of the first ramp member 92, as shown in FIG. 4. Similarly, the lower surfaces 96 of the first and second guide members 87a and 87b transition into first and the second ramp guides (not shown) of the second ramp member 94.

The first and second ramp members 92 and 94 are angled with respect to the longitudinal axis L of the device as indicated by angles α₁ and α₂, respectively. As explained further below with respect to FIGS. 7-8, the angles α₁ and α₂ cause the inner and outer segments 30 and 50 of the clip device 20 to become spaced apart from one another in a tissue receiving configuration by a desired angular amount. A physician may select a delivery device 80 having first and second ramp members 92 and 94 with desired angular configurations as needed for a particular procedure and to create the desired spacing between the inner and outer segments 30 and 50. For example, in the embodiment of FIGS. 3-4, each of the angles α₁ and α₂ are about 20-40 degrees with respect to the longitudinal axis L. However, greater or lesser angular degrees may be employed, and the angles α₁ and α₂ need not comprise similar degrees. For example, the angle α₁ may be about 45 degrees relative to the longitudinal axis L while the α₂ may be about 10 degrees relative to the longitudinal axis L.

Referring now to FIG. 5, an alternative delivery device 80′ is similar to the delivery device 80, with a main exception that one ramp member 92′ is provided. The upper surfaces 95 of the first and second guide members 87a and 87b transition into the ramp member 92′, which has spaced apart ramp guides similar to the first and second ramp guides 92a and 92b shown in the embodiment of FIG. 3. In operation, deployment of the clip device 20 using the alternative delivery device 80′ is similar to the delivery device 80, except that a distal region of the inner segment 30 assumes an angle α₃ relative to the longitudinal axis L, while the outer segment 50 exits the delivery device 80′ substantially parallel to the longitudinal axis L.

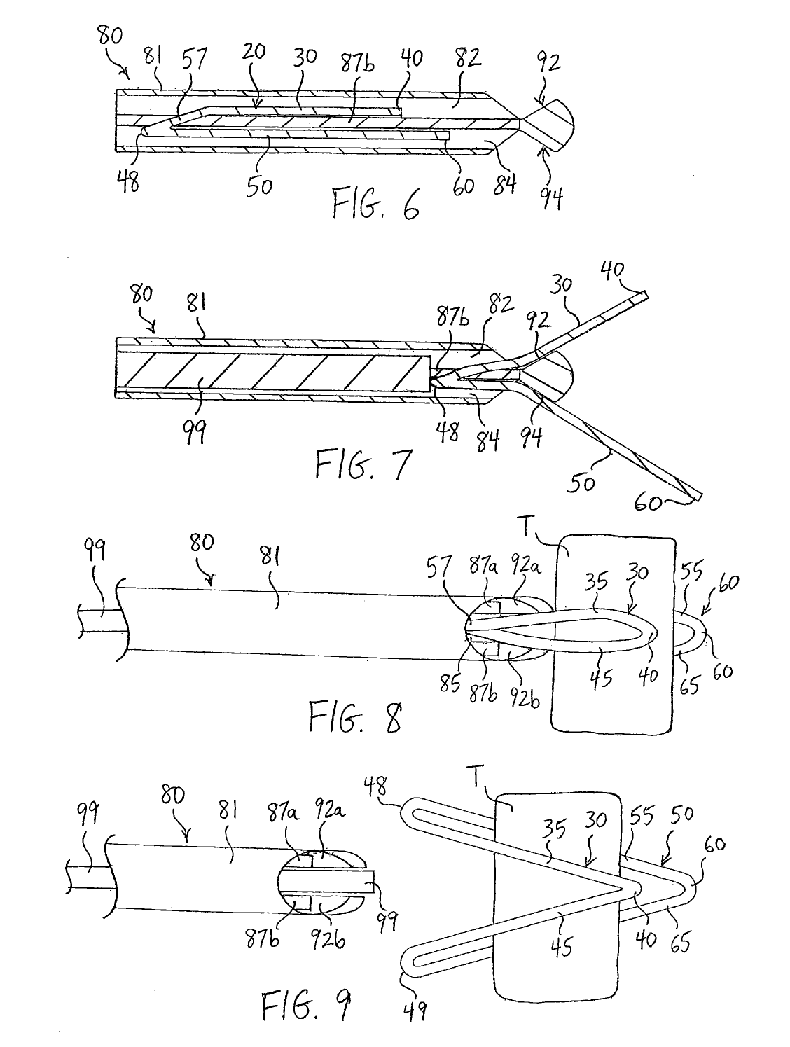

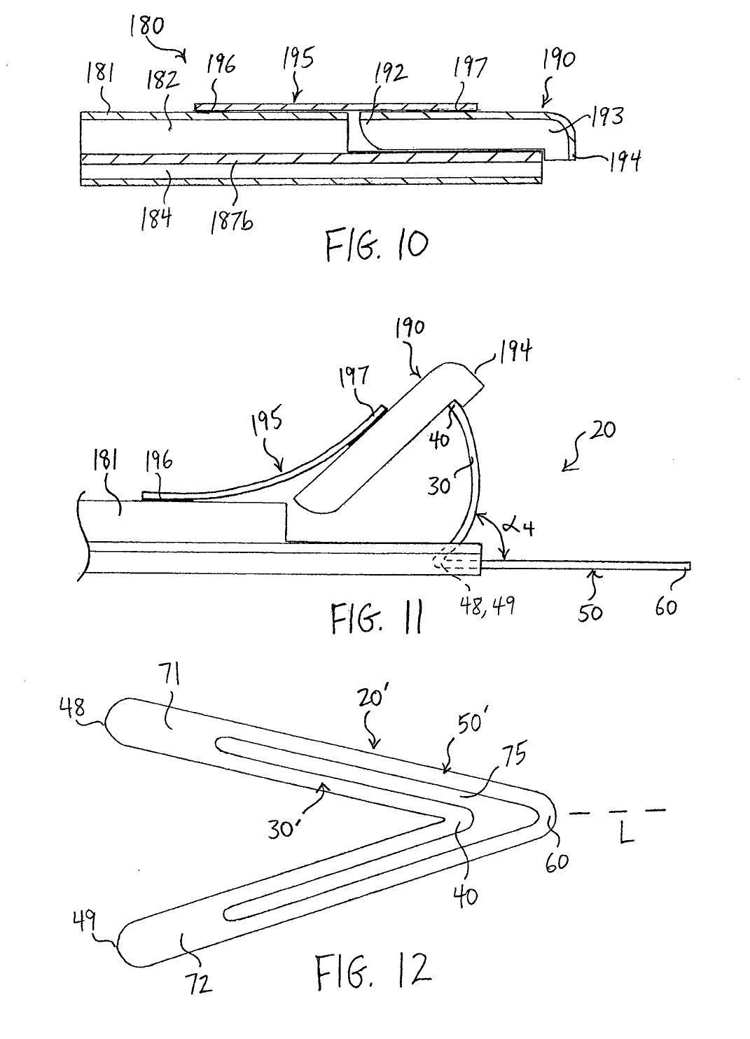

Referring now to FIGS. 6-9, exemplary method steps are described for delivering and deploying the clip device 20 of FIGS. 1-2 using the delivery device 80 of FIGS. 3-4. It should be noted that for clarity and illustrative purposes, side-sectional views of components are shown in FIGS. 6-7, while top views of components are shown FIGS. 8-9.

An exemplary procedure in which the clip device 20 may be used is a cholecystectomy, in which a physician may occlude the common bile duct and artery towards the base of the gallbladder to prevent any leakage of bile into the peritoneum. Such tissue that may need to be compressed or occluded is generally labeled as tissue T in FIGS. 8-9. However, as will be apparent, the clip device 20 may be used in various procedures in which it may be desirable to impose a compressive or occluding force upon tissue. Moreover, the delivery device 80 with the clip device 20 loaded therein may be delivered through a trocar during a laparoscopic procedure, or alternatively, the delivery device 80 may be delivered through a lumen of an endoscope during a translumenal procedure in which the endoscope may be inserted through a visceral wall, such as the stomach wall. Still further modalities are contemplated for delivering the clip device 20 in proximity to desired target tissue.

In FIG. 6, the clip device 20 is provided in the delivery configuration within the delivery device 80. As noted in FIG. 2 above, the first and second proximal apices 48 and 49 may be moved relative to one another to create an overall reduced width w₁ (see FIG. 2) of the clip device 20 that is slightly smaller than an inner diameter d₁ (see FIG. 3) of the tubular member 81.

The clip device 20 is loaded into the delivery device 80 such that the first and second proximal apices 48 and 49 are positioned within the second pathway 84 of the tubular member 81. Further, the entirety of the outer segment 50, including the distal apex 60, is positioned within the second pathway 84, as depicted in FIG. 6. By contrast, a distal portion of the inner segment 30, including the distal apex 40, is positioned within the first pathway 82 of the tubular member 81. Notably, the narrowed region 57 of the inner segment 30 extends between the first and second pathways 82 and 84 via the lumen 85, thereby allowing portions of the inner and outer segments 30 and 50 to be positioned above and below the guide members 87a-87b, respectively.

When in the delivery configuration, a portion of the first and second regions 35 and 45 of the inner segment 30 are disposed above and optionally may ride along the upper surfaces 95 of the first and second guide members 87a and 87b, respectively. Further, the first and second regions 55 and 65 of the outer segment 50 are disposed below and optionally may ride along the lower surfaces 96 of the first and second guide members 87a and 87b, respectively, as depicted in FIG. 6. The lateral width of the guide members 87a and 87b is sufficient to engage at least a portion of the inner segment 30.

Referring now to FIGS. 7-8, with the clip device 20 loaded into the delivery device 80 as explained above, a stylet 99 may be distally advanced through the lumen 85, first pathway 82 and/or second pathway 84 to cause a corresponding distal advancement of the clip device 20. As the clip device 20 is advanced distally, the distal apex 60 of the outer segment 50 will be directed in a radially outward direction, i.e., downward with respect to the page in the z-dimension, by the second ramp member 94, and the distal apex 40 of the inner segment 30 will be directed in a radially outward direction, i.e., upward with respect to the page in the z-dimension, by the first ramp member 92 to assume the tissue receiving configuration, as shown in FIG. 7.

In the tissue receiving configuration, a distal region of the inner segment 30 assumes the angle α₁ relative to the longitudinal axis L, and a distal region of the outer segment 50 assumes the angle α₂ relative to the longitudinal axis L, as explained in FIG. 4 above. If each angle α₁ and α₂ is about 30 degrees, then a 60 degree spacing is formed between the distal regions of the inner and outer segments 30 and 50. In this state, the delivery system 80 may be maneuvered such that the tissue T is disposed at least partially between the inner and outer segments 30 and 50, as shown in FIG. 8. Notably, the clip device 20 advantageously may partially surround the tissue T, but may be repositioned with respect to the tissue before being finally deployed by adjusting the position or orientation of the tubular member 81, thereby reducing the likelihood of an inaccurate positioning of a deployed clip device.

Referring now to FIG. 9, in a next step, further distal advancement of the stylet 99 causes ejection of the clip device 20 from the delivery device 80. As noted above, the clip device 20 is inclined to return towards the unbiased configuration shown in FIG. 1 whereupon the proximal apices 48 and 49 move outwardly in the y-dimension, i.e., laterally apart. In the deployed configuration, the inner and outer segments 30 and 50 impose a compressive force upon the tissue T, as depicted in FIG. 9. After the clip device 20 has been deployed, the delivery device 80 may be removed from the patient, or alternatively, one or more additional clips may be deployed in a similar manner.

In one technique, multiple clip devices 20 may be loaded in a serial manner within the delivery device 80, such that the stylet 99 is in contact with a proximal-most clip device 20. In use, distal advancement of the stylet 99 urges each of the clip devices 20 distally simultaneously, and the clip devices 20 may be ejected one at a time based on distal to proximal loading sequence of the clip devices.

Advantageously, the clip device 20 may be delivered via a relatively low profile insertion device, such as a trocar or endoscope, and then deployed to a relatively wide profile having an increased surface area engagement with tissue. For example, by bringing the first and second proximal apices 48 and 49 adjacent to one another, or overlapping one another, the width of the clip device may be reduced by a factor of about 3-5 times in the delivery configuration relative to the deployed configuration. As one clip device 20 may engage a relatively large surface area of tissue, fewer clip devices may need to be deployed, resulting in shorter operating times and potentially eliminating the need for multiple incisions.

Referring now to FIGS. 10-11, an alternative delivery device 180 suitable for delivering the clip device 20 is shown from side-sectional and side views, respectively. Like the delivery device 80 described above, the delivery device 180 comprises a tubular member 181 having proximal and distal regions, and first and second guide members separated by a lumen extending therebetween. It should be noted that, in FIG. 10, only one guide member 187b is shown from the side-sectional view, however the first and second guide members are disposed about 180 degrees apart around the inner circumferences of the tubular member 181, in the manner described above for the guide members 87a and 87b of the delivery device 80. Alternatively, only a single guide member may be provided and used to separate the distal apices 40 and 60 into the tissue receiving configuration.

The loading of the clip device 20 into the delivery device 180 is similar to the loading process described in FIG. 6 above, wherein the clip device 20 is loaded in the delivery configuration within the delivery device 180 such that the first and second proximal apices 48 and 49, and the entirety of the outer segment 50 including the distal apex 60, are positioned within the second pathway 184 of the tubular member 81. Further, a distal portion of the inner segment 30, including the distal apex 40, is positioned within the first pathway 182 of the tubular member 181.

In the embodiment of FIGS. 10-11, the first and second ramp members 92 and 94 described above are omitted, and a deflectable tip 190 is provided. The deflectable tip 190 has proximal and distal ends 192 and 194 and a hollow interior space 193, as shown in FIG. 10. The deflectable tip 190 is spaced apart and coupled to the tubular member 181 using a flexible connector 195, which may be formed from a bendable material such as plastic, stainless steel, a shape-memory alloy, or another suitable material. Alternatively, the deflectable tip 190 may be coupled to the tubular member 181 using a hinge that allows relative rotation of the deflectable tip 190, as shown in FIG. 11. The distal end 194 of the deflectable tip 190 is radially located in line with the pathway 182.

In use, the clip device 20 is advanced distally within the delivery device 180 by the stylet 99 described in FIGS. 7-9 above. During advancement, the inner and outer segments 30 and 50 are positioned at least partially within the first and second pathways 182 and 184, respectively. As the clip device 20 approaches the distal region of the delivery device 180, the distal tip 60 of the outer segment 50 extends distally from the second pathway 184, while the distal tip 40 of the inner segment 30 catches on the deflectable tip 190. As the stylet 99 is further advanced, the distal end 194 of the deflectable tip 190 temporarily retains the inner segment 30 such that both components deflect away from the longitudinal axis L, as shown in FIG. 11. In this tissue receiving configuration, a distal region of the inner segment 30 assumes the angle α₄ relative to the longitudinal axis L and the outer member 50. Notably, in this embodiment, the outer member 50 exits the delivery device 180 substantially parallel to the longitudinal axis L. In this tissue receiving configuration, the delivery system 180 may be maneuvered such that tissue is disposed at least partially between the inner and outer segments 30 and 50, as described with respect to FIGS. 7-8 above.

Subsequently, further distal advancement of the stylet 99 causes ejection of the inner segment 30 from the interior space 193 of the deflectable tip 190, such that the inner segment 30 is no longer constrained and is biased to return to the unbiased configuration shown in FIG. 1. In the deployed configuration, the inner and outer segments 30 and 50 impose a compressive force upon the tissue, as described in FIG. 9 above.

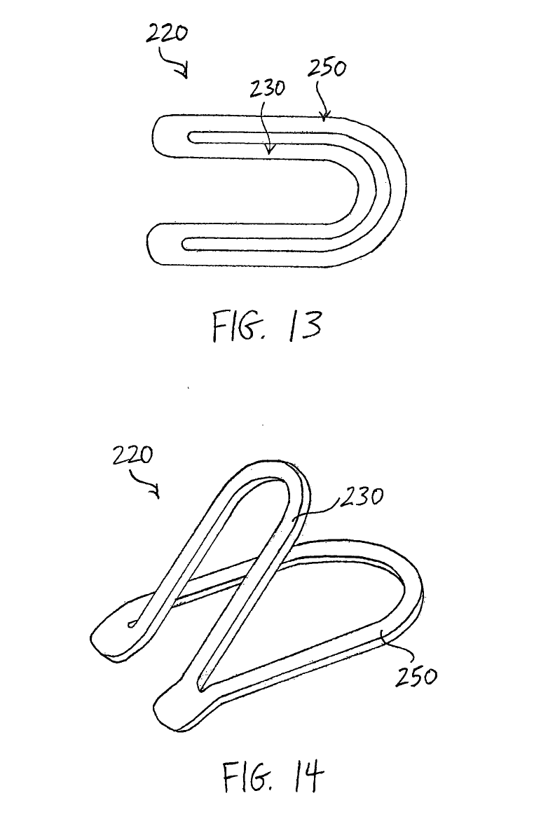

Referring to FIG. 12, an alternative clip device 20′ is shown that may be suited for use with the delivery device 180 of FIGS. 10-11. The clip device 20′ is substantially identical to the clip device 20 described above, with the main exception that solid proximal segments 71 and 72 separate inner and outer segments 30′ and 50′. Advantageously, when used with the delivery device 180 of FIGS. 10-11, the solid proximal segments 71 and 72 may increase the time that the clip device 20′ may be retained within the delivery device 180, which may ensure that the inner segment 30′ opens fully before the clip device 20 is ejected, as depicted in FIG. 11. In effect, the solid proximal segments 71 and 72 help hold the clip device 20′ within the delivery device 180 longer to ensure the desired angle α₄ may be achieved prior to deployment.

Referring now to FIGS. 13-14, an alternative embodiment of a clip device 220 is shown from a top view in an unbiased configuration and from a perspective view in a tissue receiving configuration. The clip device 220 is similar to the clip device 20 described above with the main exception that the inner and outer segments 230 and 250 are U-shaped, instead of the respective V-shaped inner and outer segments 30 and 50 described above. The loading and deployment of the clip device 220 may be identical to the loading and deployment of the clip device 20, as described above.

While various embodiments of the invention have been described, the invention is not to be restricted except in light of the attached claims and their equivalents. Moreover, the advantages described herein are not necessarily the only advantages of the invention and it is not necessarily expected that every embodiment of the invention will achieve all of the advantages described.

We claim:

1. A clip device for use in a medical procedure, the clip device having three dimensions including longitudinal, lateral and vertical dimensions, the clip device comprising:
an inner segment; and
an outer segment, wherein the inner segment is generally nestled at least laterally within the outer segment in an unbiased configuration,
wherein the clip device further has a delivery configuration, a tissue receiving configuration and a deployed configuration,
wherein at least a portion of the inner and outer segments are movable in the lateral dimension to achieve a reduced profile in the delivery configuration, and
wherein a distal region of the outer segment is movable with respect to a distal region of the inner segment in the vertical dimension, thereby creating a spacing between the inner and outer segments for surrounding tissue in the tissue receiving configuration.

2. The clip device of claim 1 wherein the clip device comprises at least two proximal apices that are capable of movement towards one another in the lateral dimension in the delivery configuration.

3. The clip device of claim 1 wherein the inner and outer segments comprise generally identical shapes in the unbiased configuration, wherein the outer segment is proportionally larger than the inner segment.

4. The clip device of claim 3 wherein the inner segment and the outer segment each comprise V shapes in the unbiased configuration.

5. The clip device of claim 4 wherein the inner and outer segments each comprise first and second regions separated by apices, wherein a slit separates the first region of the outer segment from the first region of the inner segment, and further separates the second region of the outer segment from the second region of the inner segment.

6. The clip device of claim 5 wherein the first region of the outer segment and the first region of the inner segment are movable laterally towards the second region of the outer segment and the second region of the inner segment to achieve the delivery configuration.

7. A system for delivering a clip device, the clip device having three dimensions including longitudinal, lateral and vertical dimensions, the system comprising:
a delivery device comprising a tubular member having first and second pathways separated by at least one guide member; and
a clip device comprising inner and outer segments, the clip device having an unbiased configuration, a delivery configuration, a tissue receiving configuration and a deployed configuration,
wherein, in the delivery configuration, the clip device is configured to be loaded within the delivery device with the inner segment positioned at least partially within the first pathway and the outer segment positioned at least partially within the second pathway, and further wherein at least a portion of the inner and outer segments are movable in the lateral dimension to achieve a reduced profile in the delivery configuration,
wherein, in the tissue receiving configuration, a distal region of the outer segment is movable with respect to a distal region of the inner segment in the vertical dimension, thereby creating a spacing between the inner and outer segments for surrounding tissue, and
wherein the inner and outer segments are biased to impose a compressive force upon tissue in the deployed configuration.

8. The system of claim 7 wherein the inner and outer segments of the clip device comprise generally identical shapes in the unbiased configuration, wherein the outer segment is proportionally larger than the inner segment, and wherein a slit separates the inner and outer segments in the unbiased configuration.

9. The system of claim 8 wherein the inner segment and the outer segment each comprise V shapes in the unbiased configuration.

10. The system of claim 7 further comprising:
first and second guide members that protrude inward from an interior wall of the tubular member are spaced apart by a lumen of the tubular member, wherein the first and second guide members are disposed about 180 degrees apart within the tubular member; and
a stylet sized for longitudinal advancement within the lumen between the first and second guide members and configured to engage a portion of the clip device.

11. The system of claim 7 wherein, in the delivery configuration, the clip device comprises a narrowed region sized to extend through the lumen of the tubular member between the first and second guide members.

12. The system of claim 7 further comprising a first ramp member positioned at a distal end of the first pathway of the delivery device, wherein the first ramp member is angled with respect to a longitudinal axis of the delivery device to guide the inner segment towards a desired angle in the tissue receiving configuration.

13. The system of claim 12 further comprising a second ramp member positioned at a distal end of the second pathway of the delivery device, wherein the second ramp member is angled with respect to the longitudinal axis of the delivery device to guide the outer segment towards a desired angle in the tissue receiving configuration.

14. The system of claim 7 wherein the delivery device comprises a deflectable tip coupled to a distal region of the tubular member by a flexible connector, wherein the deflectable tip is configured to engage a distal tip of the inner segment of the clip device to urge the inner segment at an angle with respect to a longitudinal axis of the delivery device in the tissue receiving configuration.

15. A method for delivering a clip device, the clip device having three dimensions including longitudinal, lateral and vertical dimensions, the method comprising:
providing a delivery device comprising a tubular member having first and second pathways separated by at least one guide member;
loading a clip device comprising inner and outer segments into the delivery device in a delivery configuration with the inner segment positioned at least partially within the first pathway and the outer segment positioned at least partially within the second pathway, wherein at least a portion of the inner and outer segments are movable in the lateral dimension to achieve a reduced profile in the delivery configuration;
advancing the clip device distally with respect to the delivery device to cause the clip device to achieve a receiving configuration in which a distal region of the outer segment is spaced apart from a distal region of the inner segment;
positioning an object between the inner and outer segments; and
deploying the clip device from the delivery device to allow the inner and outer segments to move towards each other and impose a compressive force upon the object in a deployed configuration.

16. The method of claim 15 wherein the inner and outer segments of the clip device comprise generally identical shapes in an unbiased configuration, wherein the outer segment is proportionally larger than the inner segment, and wherein a slit separates the outer and inner segments in the unbiased configuration.

17. The method of claim 16 wherein the inner segment and the outer segment each comprise V shapes in the unbiased configuration.

18. The method of claim 15 further comprising:
providing first and second guide members that protrude inward from an interior wall of the tubular member and are spaced apart by a lumen of the tubular member, wherein the first and second guide members are disposed about 180 degrees apart within the tubular member; and
longitudinally advancing a stylet within the lumen between the first and second guide members to selectively engage a portion of the clip device.

19. The method of claim 15 further comprising:
providing a first ramp member positioned at the distal end of the first pathway of the delivery device, wherein the first ramp member is angled with respect to a longitudinal axis of the delivery device; and
advancing the clip device distally such that the first ramp member guides the inner segment towards a desired angle with respect to the longitudinal axis in the receiving configuration.

20. The method of claim 19 further comprising:
providing a second ramp member positioned at the distal end of the second pathway of the delivery device, wherein the second ramp member is angled with respect to a longitudinal axis of the delivery device; and
advancing the clip device distally such that the second ramp member guides the outer segment towards a desired angle with respect to the longitudinal axis in the receiving configuration.
